(19) United States
(12) Patent Application Publication
(10) Pub. No.: US 2002/0169148 A1
(43) Pub. Date: Nov. 14, 2002

(54) Use of alendronate for the prevention of osteoporosis

(75) Inventor: Ashley J. Yates, Westfield, NJ (US)

Correspondence Address: MERCK AND CO INC, P O BOX 2000, RAHWAY, NJ, 070650907

(73) Assignee: Merck & Co., Inc.

(21) Appl. No.: 10/114,287

(22) Filed: Apr. 2, 2002

Related U.S. Application Data
(63) Continuation of application No. 09/794,722, filed on Feb. 27, 2001.
Application No. 09/794,722 is a continuation of application No. 08/952,830, filed on Nov. 25, 1997, now abandoned.
Application No. 08/952,830 is a 371 of international application No. PCT/US96/07912, filed on May 29, 1996.

(51) Int. Cl. A61K 31/66

(52) U.S. Cl. 514/102

(57) ABSTRACT

Alendronate, an aminobisphosphonate, can prevent osteoporosis in early post menopausal women.

### FIELD OF THE INVENTION

This invention relates to the use of alendronate, an aminobisphosphonate, for the prevention of osteoporosis in early postmenopausal women.

### BACKGROUND OF THE INVENTION

Alendronate, 4-amino-1-hydroxybutylidene-1,1-bisphosphonic acid, and its pharmaceutically acceptable salts has been found to be useful in the treatment of osteoporosis. Alendronate is a specific inhibitor of bone resorption. It has a high affinity for bone mineral and is taken up into the bone selectively where it inhibits osteoclast activity. While alendronate has been shown to be useful in restoring lost bone, there has been no indication that it can prevent the loss of bone in otherwise healthy individuals.

Peak bone mass in women is achieved at around 30-35 years of age, after which bone mass is lost progressively throughout life. The rate of loss is accelerated during the early post menopausal period, especially at sites with a high component of trabecular bone.

The average woman probably has a greater than 40% chance of developing at least one osteoporotic fracture during her lifetime. Osteoporotic fractures, especially of the hip, are associated with a marked reduction in the quality of life and high cost of treatment. The total costs and morbidity associated with all osteoporotic fractures are certain to substantially exceed those of hip fracture alone, although precise estimates are not available.

At the present time, the only approved therapy for prevention of osteoporosis is estrogen replacement therapy. Along with a prevention of bone loss associated with reduced endogenous estrogen production, administration of estrogen can help reduce post menopausal symptoms such as vasomotor instability, vaginal atrophy, and an improvement in the lipid profile with a probable reduction in cardiovascular problems. However, at the doses commonly employed for bone loss prevention, many women lose bone despite continued treatment. Further, estrogen treatment is also associated with some serious risks, including endometrial carcinoma, symptomatic gall bladder disease, and a possible increase in the incidence of breast cancer. Although some of these risks can be lowered by addition of progestins to the therapeutic regimen or by yearly endometrial biopsies, a large proportion of women will not accept long-term estrogen treatment mainly because of poor tolerability and safety concerns.

It would be desirable to have an agent which can prevent osteoporosis which does not have the risks and possible side effects associated with estrogen.

### DESCRIPTION OF THE INVENTION

This invention relates to a method of preventing osteoporosis in women having a normal bone mineral density comprising administering a prophylactically effective amount of alendronate or a pharmaceutically acceptable salt thereof for a sufficient amount of time.

A further aspect of this invention is a method of reducing the risk of fracture in women by administering a prophylactically effective amount of alendronate or a pharmaceutically acceptable salt thereof for a substantial period of time.

Yet another aspect of this invention is a method of preventing osteoporosis in early postmenopausal women by administering a prophylactically effective amount of alendronate or a pharmaceutically acceptable salt thereof.

In the absence of preventive treatment, the microstructure of the bone deteriorates as bone loss progresses, leading to a decrease in bone strength per unit bone mass. Prophylactic administration of alendronate has been found, in accordance with this invention, to preserve normal microstructure and normal bone strength. Thus a further aspect of this invention is a method of preserving normal bone microstructure and bone strength by administering a prophylactically effective amount of alendronate or a pharmaceutically acceptable salt thereof.

As used throughout the specification and claims, the following definitions will apply:

“Prophylactically effective amount”: an amount of alendronate or a pharmaceutically acceptable salt thereof which is sufficient to prevent osteoporosis in women not currently suffering from osteoporosis. This amount may or may not be a pharmaceutically acceptable amount, i.e. sufficient to treat osteoporosis, i.e. restore bone mass in a patient who is currently suffering from osteoporosis.

“Substantial period of time”: a sustained period, i.e. at least about three years, and preferably longer.

“Osteoporosis”: a condition wherein a person's bone mineral density is more than about 2 standard deviations below the peak bone mineral density.

“Early post-menopause”: less than approximately five years after a woman's menstrual periods have ceased.

Alendronate may be prepared according to any of the processes described in U.S. Pat. Nos. 5,019,651, 4,992,007, and U.S. application Ser. No. 08/286,151, filed Aug. 4, 1994, each of which is hereby incorporated by reference. The pharmaceutically acceptable salts of alendronate include salts of alkali metals (e.g., Na, K), alkali earth metals (e.g. Ca), salts of inorganic acids, such as HCl and salts of organic acids such as citric acid and amino acids. Sodium salt forms are preferred, particularly the monosodium salt trihydrate form.

The compounds of the present invention can be administered in oral dosage forms such as tablets, capsules (each of which includes sustained release or timed release formulations), pills, powders, granules, elixirs, paste, tinctures, suspensions, syrups, emulsions, and zydis. Likewise they may be administered in an intravenous (bolus or infusion), intraperitoneal, subcutaneous, or intramuscular form, all using forms well known to those of ordinary skill in the pharmaceutical arts. An effective but non-toxic amount of the compound desired can be used as a osteoporosis-preventing agent.

The dosage regimen utilizing the claimed method is selected in accordance with a variety of factors including age, weight, sex, and medical condition of the patient; the route of administration; the renal and hepatic function of the patient; and the particular compound or salt thereof employed. An ordinarily skilled physician or clinician can readily determine and prescribe the effective amount of the drug required to prevent osteoporosis.

Oral dosages of the present invention will range from between 0.05 mg per kg of body weight per day (mg/kg/day) to about 1.0 mg/kg/day. Preferred oral dosages in humans may range from daily total dosages of about 2.5-20 mg/day over the effective treatment period, and a preferred prophylactic amount is 2.5, 5, or 10 mg/day.

Alendronate may be administered in a single daily dose or in a divided dose. It is desirable for the dosage to be given in the absence of food, preferably from about 30 minutes to 2 hours prior to a meal, such as breakfast, to permit adequate absorption.

In the methods of the present invention, the active ingredient is typically administered in admixture with suitable pharmaceutical diluents, excipients or carriers (collectively referred to herein as “carrier materials”) suitably selected with respect to the intended form of administration, i.e. oral tablets, capsules, elixirs, syrups and the like and consistent with conventional pharmaceutical practices. For example, for oral administration in the form of a tablet or capsule, the active ingredient can be combined with an oral, non-toxic, pharmaceutically acceptable inert carrier such as lactose, starch, sucrose, glucose, methyl cellulose, cros-carmellose sodium, magnesium stearate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture of active ingredient(s) and inert carrier materials. Suitable binders may include starch, gelatin, natural sugars such as glucose, anhydrous lactose, free-flow lactose, beta-lactose, and corn sweeteners, natural and synthetic gums, such as acacia, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. A particularly preferred tablet formulation is that described in U.S. Pat. No. 5,358,941, which is hereby incorporated by reference.

The compounds used in the instant method may also be coupled with soluble polymers as targetable drug carriers. Such polymers can include polyvinylpyrrolidone, pyran co-polymer, polyhydroxylpropyl-methacrylamide and the like.

The following non-limiting examples are presented to illustrate the invention.

### EXAMPLE 1

Women enrolled in this study are in good general health and are between 45-59 years old and have been selected randomly from a target population who live in a defined geographical area. The majority are early postmenopausal. Fewer than 15 percent of the participants have any incidence of osteoporosis evident on baseline spinal dual-energy X-ray densitometry.

Each subject is randomized to ether placebo, alendronate low dose (ALN 2.5 mg per day), alendronate high dose (ALN 5 mg per day) or open labeled estrogen/progestin (E/P). The estrogen/progestin group (in the United States) will receive the conjugated estrogen PREMARIN® (0.625 mg per day) and the medroxyprogesterone acetate PROVERA® (5 mg per day) taken continuously throughout the calendar month. Outside the United States, the estrogen/progestin group will receive micronized 17b-estradiol and norethisterone acetate (Trisequens) as a cyclical regimen. All subjects who have a calcium intake of less than 500 mg per day will be advised to increase their calcium intake (either by diet or supplements) to above this level. Distribution of the groups is shown in TABLE 1. The duration of treatment in each of the groups is given in TABLE 2.

TABLE 1
TREATMENT GROUPS

| | | STRATUM 1 | STRATUM 2 | |
|---|---|---|---|---|
| GROUP | TREATMENT | N | N | TOTAL |
| A | Placebo | 150 | 300 | 450 |
| B | ALN 2.5 mg | 150 | 300 | 450 |
| C | ALN 5 mg | 150 | 300 | 450 |
| D | E/P | 150 | — | 150 |
| TOTAL | | 600 | 900 | 1500 |

ALN = Alendronate
E/P = Estrogen/Progestin

TABLE 2
STUDY SCHEMA

| | | YEAR OF STUDY | | |
|---|---|---|---|---|
| GROUP | N | 1 and 2 | 3 and 4 | 5 and 6 |
| A | 450 | Placebo | Placebo | ALN OD; or |
| | | | | Placebo* |
| B1 | 150 | ALN 2.5 mg | ALN 2.5 mg | ALN 2.5 mg |
| B2 | 150 | ALN 2.5 mg | ALN 2.5 mg | Placebo |
| B3 | 150 | ALN 2.5 mg | Placebo | |
| C1 | 150 | ALN 5 mg | ALN 5 mg | ALN 5 mg |
| C2 | 150 | ALN 5 mg | ALN 5 mg | Placebo |
| C3 | 150 | ALN 5 mg | Placebo | |
| D | 150 | E/P | E/P | |

ALN = Alendronate
OD = Optimal Dose (either 2.5 or 5 mg).
*Subsequent randomization for placebo group Years 5 and 6 extension
E/P = Estrogen/Progestin

The study is double blind (for women receiving either alendronate or placebo) for the first two years, at the end of which a first analysis is performed. The study remains double blind until each subject reaches the end of the fourth year of study, when the blind is broken for each subject individually. Subjects are informed only whether or not they received active treatment with alendronate, and, if so, whether they were treated for two or four years. Subjects will not be informed of the dose of the study drug. Those subjects who remain in the blinded study for years 5 and 6, and the investigators remain blinded to their treatment allocation during the extension period.

Subjects in Group “A” (See TABLE 2) continue to take blinded placebo for four years. At the end of four years these women will be informed that they had received placebo during Years 1 to 4. They are then given the option to be further randomized (1:1) between blinded placebo and alendronate and the “optimal” dose or to exit the study.

Groups B1 and C2 receive the 2.5 or 5 mg of alendronate, respectively for six years. Groups B2 and C2 will remain on the 2.5 and 5 mg of alendronate, respectively for four years before switching to placebo for the final two years of the study. Those subjects who remain in the study for Years 5 and 6 will be blinded (double blind) regarding their allocation to active drug or placebo for Years 5 and 6. Groups B3 and C3 remain on the 2.5 and 5 mg alendronate, respectively for only two years before switching to placebo for the third and fourth years of the study. They will discontinue study drug after the fourth year.

Subjects in Group D continue the open-label estrogen/progestin treatment for four years, after which they will discontinue the study drug after the fourth year.

After four years, women receiving alendronate are not developing signs of osteoporosis, as measured, e.g. by decreases in bone mineral density, whereas those receiving placebo are experiencing a loss in bone mineral density. The differences are statistically significant.

What is claimed is:

1. A method of preventing osteoporosis in early postmenopausal women comprising administering a prophylactically effective dose of alendronate or a pharmaceutically effective salt thereof.

2. A method according to claim 1 wherein the alendronate is administered orally.

3. A method according to claim 2 wherein the alendronate is administered once a day.

4. A method according to claim 3 wherein the salt of alendronate is monosodium salt trihydrate.

5. A method according to claim 4 wherein the dose is 2.5 to 20 mg/day.

6. A method according to claim 5 wherein the dose is selected from the group consisting of 2.5, 5, and 10 mg/day.

7. A method of preventing osteoporosis in early postmenopausal women comprising administering 2.5 to 20 mg/day of alendronate monosodium salt trihydrate.
